(19) United States
(12) Patent Application Publication
(10) Pub. No.: US 2002/0081654 A1
(43) Pub. Date: Jun. 27, 2002

(54) Targeting hydrolase enzymes

(76) Inventors: Mauro Sergio Sandrin, Brunswick (AU); Ian Farquhar Campbell McKenzie, Brunswick (AU); Narin Osman, Heidelberg (AU)

Correspondence Address: MERCHANT & GOULD PC, P.O. BOX 2903, MINNEAPOLIS, MN, 55402-0903, US

(21) Appl. No.: 09/828,707

(22) Filed: Apr. 6, 2001

Related U.S. Application Data
(60) Provisional application No. 60/195,656, filed on Apr. 7, 2000.

(51) Int. Cl. C12P 21/02

(52) U.S. Cl. 435/69.1; 435/320.1

(57) ABSTRACT

A nucleic acid construct encoding a peptide with hydrolase activity and a heterologous localization signal which locates the peptide, upon expression of the construct in a cell, to a cellular location proximal to the cellular site of a glycosylated agent or a glycosylating agent. The construct can be used to produce, for example, organs suitable for transplantation which show reduced levels of the epitope Galα(1,3)Gal.

### FIELD OF THE INVENTION

The present invention relates to a nucleic acid construct and a method of locating a peptide with hydrolase activity in a cell where it is proximal to glycosylated agents or glycosylation enzymes. The nucleic acid construct and method of the invention can be used in the production of xenogenic organs for transplantation or in gene therapy where it is desirable to reduce the amount of glycosylation of certain molecules.

### BACKGROUND OF THE INVENTION

α-Galactosidase (E.C. 3.2 1.22) is a lysosomal glycosidase which—hydrolyses terminal α-galactose residues from glycoconjugates. Previously, the inventors have reported decreased levels of cell surface Galα(1,3)Gal in transgenic mice expressing human α-galactosidase. It is likely that by eliminating Galα(1,3)Gal, the major xenoepitope responsible for hyperacute rejection in pig-primate transplants, xenotransplantation will be a viable alternative for transplant recipients. From the inventors' earlier studies it was clear that to achieve maximal reduction of Galα(1,3)Gal, and subsequently maximal success in xenotransplantation, α-galactosidase needs to be in close proximity to newly synthesized molecules containing terminal galactose residues. They hypothesized that by targeting α-galactosidase to the transgolgi network (TGN) it might enable removal of the Galα(1,3)Gal epitope prior to its journey to the cell surface and thereby produce cell surface membranes with reduced levels of the xenoepitope

In work leading to the present invention, the inventors have surprisingly found that enzymes with hydrolase activity can be located in the TGN or at the surface of the cell such that they are proximal to newly synthesized, glycosylated molecules and/or glycosylation enzymes. This was achieved by producing a nucleic acid construct encoding the hydrolase enzyme and an appropriate signal which locates the enzyme at the desired cellular location.

Specifically, the inventors found that targeting α-galactosidase, which is a soluble enzyme, to the Golgi network could be achieved by attaching a transmembrane region and a Golgi localization signal to the α-galactosidase molecule. In this work, the inventors used the transmembrane region and localization signals contained within furin, a trans Golgi resident, Type I protein, Localization signals of this protein have been well defined using reporter molecules and deletion mutants (Molloy 1994, Bosshart 1994, Schafer 1995, Jones 1995).

The construction and use of chimeras containing targeting signals found within the cytoplasmic domain of furin to localize α-galactosidase to the TGN and cell surface membrane are described. The inventors provide morphological evidence that chimeric α-galactosidase co-localized with several marker proteins for the TGN, and biochemical evidence that the chimeras are enzymatically active at their new cellular locations. These findings reveal that lysosomal glycosidases can be redirected to alternative cellular destinations without compromising enzyme activity.

### SUMMARY OF THE INVENTION

Thus, in a first aspect, the present invention provides a nucleic acid construct encoding a peptide with hydrolase activity and a heterologous localization signal which locates the peptide, upon expression of the construct in a cell, to a cellular location proximal to the cellular site of a glycosylated agent or a glycosylating agent.

The glycosylated agent is preferably a newly synthesized molecule comprising at least one sugar moiety. More preferably, the agent is a newly synthesized molecule comprising terminal galactose residues, particularly an epitope. In a particularly preferred embodiment, the terminal galactose residues are α-linked gal.

The glycosylating agent may be a glycosylating enzyme such as a galactosyl transferase, more preferably α1,3 galactosyl transferase (a Type II membrane protein of the TGN which adds terminal galactose to glycoproteins).

The term “nucleic acid” may be DNA, RNA, single stranded, double stranded or covalently closed circular or linear.

The term “peptide” refers to an amino acid chain with a required number of amino acids to provide the relevant functional activity or to a polypeptide or protein.

The term peptide with “hydrolase activity” is a peptide which is able to cleave sugars, particularly galactosyl residues on another molecule. Preferably, the peptide is a deglycosylase such as galactosidase or a functional portion thereof able to cleave by hydrolysis a terminal sugar moiety from a glycosylated molecule. More preferably, the galactosidase removes the terminal, α-linked galactose from Galα(1,3)Gal.

The term “localization signal” is an amino acid sequence able to localize the peptide at the desired location within the cell. The localization signal may be based on any suitable localization signal such as that derived from proteins resident in organelles to which the peptide is desired to be targeted. Preferably, the signal is based on an endosomal or lysosomal resident protein or a portion or fragment thereof effective to localise the peptide to a desired target site. It may also be one based on a cell surface resident protein. Most preferably, the signal is based on a protein resident in the Golgi or the transgolgi network. Preferably, the localization signal is attached to the carboxy terminus of the peptide.

The nucleic acid construct is expected to be useful in producing organs or tissues suitable for transplantation where the level of undesirable epitopes present on the cell surface may be reduced. In particular, it is envisaged that the level of Galα(1,3)Gal on the surface of cells will be reduced as this is a major xenoepitope responsible for hyperacute rejection in, for example, pig-primate transplants. To achieve this end, it may be necessary to select an appropriate gene encoding the hydrolase or deglycosylation enzyme for inclusion in the construct. In particular, it may be necessary to select a gene encoding an enzyme with biochemical characteristics appropriate to the intended location of the peptide within the cell. For example, if the intended location is the TGN then the hydrolase should be one that can function in that environment.

The nucleic acid construct may be produced according to standard recombinant DNA techniques such as those described in Sambrook et al

In a second aspect, the present invention provides a method of locating a peptide with hydrolase activity to a cellular location proximal to the cellular site of a glycosylated agent or a glycosylating agent within a cell, said method comprising expressing in the cell a nucleic acid construct encoding a peptide with hydrolase activity and a localization signal which signal locates the peptide proximal to said glycosylated or glycosylating agent

Preferably, the peptide with hydrolase activity is a peptide which is able to cleave sugars, particularly galactosyl residues. More preferably, the peptide is a galactosidase or a functional portion thereof which is able to cleave α1,3 galactosyl residues thereby removing unwanted epitopes from the cell. More preferably, the method results in a reduction in the level of unwanted galactosyl epitopes on the cell surface.

In a third aspect, the present invention provides a cell, organ or animal in which an enzyme with hydrolase activity is located at a cellular location proximal to the cellular site of a glycosylated agent or a glycosylating agent in the cell or the cells of the organ or animal, wherein the cell or the cells express a nucleic acid construct encoding a peptide with hydrolase activity and a localization signal which signal locates the peptide proximal to said glycosylated or glycosylating agent, resulting in lowered levels of glycosylation on proteins produced by the cell or cells.

Preferably, the peptide with hydrolase activity is able to cleave sugars, particularly galactosyl residues. More preferably, the peptide comprises α-galactosidase or a functional part thereof.

Preferably, the localization signal is derived from a Type 1 Golgi resident protein or furin. However, any region of a protein which can target or localise a peptide having hydrolase activity to a site in which the peptide can function so as to hydrolyse a glycosylated or glycosylating agent is contemplated. These include but are not limited to proteins resident in lysosomes, endosomes, the RER, SER and/or the golgi or transgolgi network. The cytoplasmic or transmembrane regions of these proteins may be used, so long as they are capable of localising the peptide so that it can function in accordance with the invention.

Preferably, the cell or organ is non-primate in origin, more preferably derived from mouse, pig or sheep or other domestic animal.

Preferably, the animal is a non-primate of suitable species such as mouse, pig, sheep or dog or other domestic animal.

In a fifth aspect, the present invention provides a method of modulating hydrolysis of a glycosylated product expressed in a cell, said method comprising the step of locating a peptide as described in accordance with the second aspect of this invention.

Thus, in one embodiment, the method comprises introducing into the cell a construct in accordance with the first aspect of the present invention.

The construct may be used to target an enzyme to a site wherein the peptide can hydrolyse the glycosylated product. Thus, the site may be an endosome and the peptide may be α-galactosidase, which will deglycosylate products having α-linked terminal galactose residues. The localization signal in such a case may be based on targeting signals of proteins resident in the endosomes. Preferably, the galactosidase in such a case is a pH-modified galactosidase. Galactosidase enzymes which function at different pH are well known (e.g. coffee bean α-galactosidase). They can also be produced-by altering one or more amino acids in the enzyme (e.g. by site-directed mutagenesis) and testing for function at various pH.

In a sixth aspect, the present invention provides a method of alleviating a disease or condition in a patient caused by the presence of undesirable glycosylated products, said method comprising introducing into cells of the patient the nucleic acid construct, wherein said nucleic acid encodes a peptide with hydrolase activity and a localization signal wherein the localization signal locates the peptide to a cellular location in the cells of the patient proximal to the cellular site of said glycosylated agent or glycosylating agent producing said glycosylated products such that said hydrolase activity reduces the glycosylation on the glycosylated products.

Preferably the disease or condition is one in which the cause is unwanted accumulation of glycosylated products such as glycosylated amides, lipids and/or phospholipids. In particular, the disease is Fabry disease.

### DETAILED DESCRIPTION OF PREFERRED EMBODIMENT

### BRIEF DESCRIPTION OF THE FIGURES

FIG. 1. Schematic diagram of the α-galactosidase constructs.

Shown are wild-type α-galactosidase (HUG) and the chimera HUGCD7Fur (HCF), comprising α-galactosidase fused at its carboxy end to a CD7 stem domain and entire furin transmembrane and cytoplasmic domains, and the chimera HUGTFur which consists of α-galactosidase fused at its carboxy end to the furin transmembrane domain.

FIG. 2. Surface expression of Gala(1,3)Gal on transfected cells.

Cell surface Galα(1,3)Gal was stained with FlTC-conjugated IB4 and cells were analyzed by flow cytometry. Mock-transfected cells and cells transfected with α-galactosidase (A) and CD48 (B) are shown. Profiles are representative of those obtained in at least five experiments.

FIG. 3. Internal localization of α-galactosidase in transfected COS cells.

Cells were transfected with vector alone, pCDNA (B, D, F) or α-galactosidase (A, C, F). Cells were examined for α-galactosidase staining (A, B), γ-adaptin staining (C, D) and co-localisation of α-galactosidase staining and γ-adaptin staining (E, F—green for α-galactosidase, red for adaptin; yellow staining indicates superimposition of α-galactosidase and adaptin); the scale bar represents 20 μm.

FIG. 4 Internal location of HUGCD7Fur in transfected COS cells.

A, B: α-galactosidase staining, C: γ-adaptin staining, D: WGA staining, E, G, H: colocalization of α-galactosidase (green) and γ-adaptin (red) and superimposition of α-galactosidase and γ-adaptin (yellow), F: colocalization of α-galactosidase (green) and WGA (red) and superimposition of α-galactosidase and WGA (yellow), G: cycloheximide treated and brefeldin A treated; the scale bar represents 20 μm.

FIG. 5. Surface expression of Galα(1,3)Gal on stable expressing PIECs.

Cell surface Galα(1,3)Gal was stained with FITC-IB4 and cells were analyzed by flow cytometry. Unstained Parental PIECs (--), (A & B), Parental PIECs stained with FITC-IB4 (-), (A & B), stable expressing α-galactosidase PIECs (-), (A), stable expressing HUGCD7Fur PIECs (-), (B).

FIG. 6. Analysis of α-galactosidase activity in stable expressing PIECs.

PIECs were lysed and α-galactosidase activity determined in terms of nmoles/ug protein/hr as described. The enzyme activities of parental PIECs, PIECs stable expressing α-galactosidase, stable expressing HUCCD7FUR were determined.

FIG. 7. Localization of HUGTFUR in transfected COS cells.

HUGTFur expressing cells were surface stained (A, B) with α-galactosidase (green), then permeabilized and stained with γ-adaptin as an internal reference marker (red); panels C-H show internal staining and in panels B, D, F, and H, cells were treated with cycloheximide: α-galactosidase staining (C, D), γ-adaptin staining (E,F) and co-localisation of α-galactosidase staining and γ-adaptin staining (G, H—green for α-galactosidase, red for adaptin; yellow staining indicates superimposition of α-galactosidase and adpatin); the scale bar represents 20μm.

FIG. 8. Internalization of anti-galactosidase antibody by transfected COS cells.

COS cells expressing: vector (pCDNA1) alone (A, B), (α-galactosidase (C), HUGCD7Fur (D) and HUGTFur (E) were:

stained with secondary anti-rabbit lg-FITC only (A), and

incubated for 1 hour in culture with an irrelevant primary (anti-β-COP) (B),

and then fixed, permeabilized and stained with secondary anti-mouse lg-FITC;

incubated for 1 hr in culture with the anti-galactosidase antibody and then fixed, permeabilized and stained with the secondary anti-rabbit lg FITC (C-E);

the scale bar represent 20 μm.

FIG. 9. Surface expression of Galα(1,3)Gal for co-transfected Cos-7 cells.

48 hours post-transfection COS-7 cells were surface stained with FITC-IB4 and the number of positively staining cells determined under immunofluorescence.

FIG. 10. Surface expression of Galα(1,3)Gal in transgenic mice.

Splenocytes were harvested from mice homozygous for either HT or HCF, HT heterzygotes or F1HT/HCF hybrid (grey line) mice. Epitope expression was measured by staining with FITC-IB4 and quantitating surface fluorescence by flow cytometry, using cells from non-transgenic mice as controls (-x-).

### EXAMPLE 1

### Materials and Methods

Antibodies and Cytochemical Probes.

The following reagents were obtained from commercial sources: monoclonal anti-γ-adaptin and monoclonal anti-β-COP (Sigma, St.Louis), Isolectin B4 from Bandeiraea simplicifolia, UEA I lectin from Ulex europaeus and Wheat germ agglutinin (WGA) lectin from Triticum vulgaris (Sigma, St.Louis, Mo.). A monoclonal antibody specific for CD48 (ASH1360) was generated at the Austin Research Institute. Rabbit antiserum raised against recombinant α-galactosidase was a generous gift from Dr. R. J. Desnick (Mount Sinai School of Medicine, New York). β-COP was detected with mouse monoclonal anti-β-COP (Sigma St.Louis, Mo.). Mouse monoclonal anti-γ-Adaptin (AP-1) (Sigma, St.Louis, Mo.) was used to detect γ-Adaptin. Secondary antibodies were FlTC-conjugated sheep anti-rabbit lg F(ab′)2 fragments and biotin-conjugated sheep anti-mouse lg (Silenus, Hawthorn, Australia) and streptavidin-Texas Red-X conjugate (Molecular Probes Inc., Eugene, Oreg.).

Generation of α-galactosidase constructs.

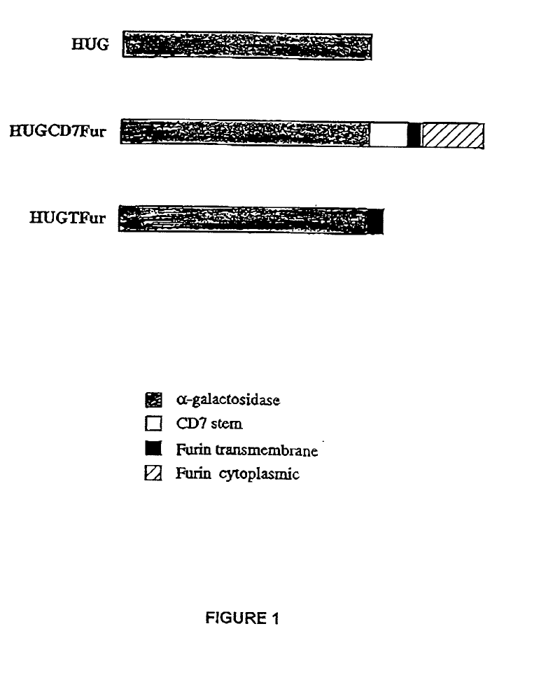

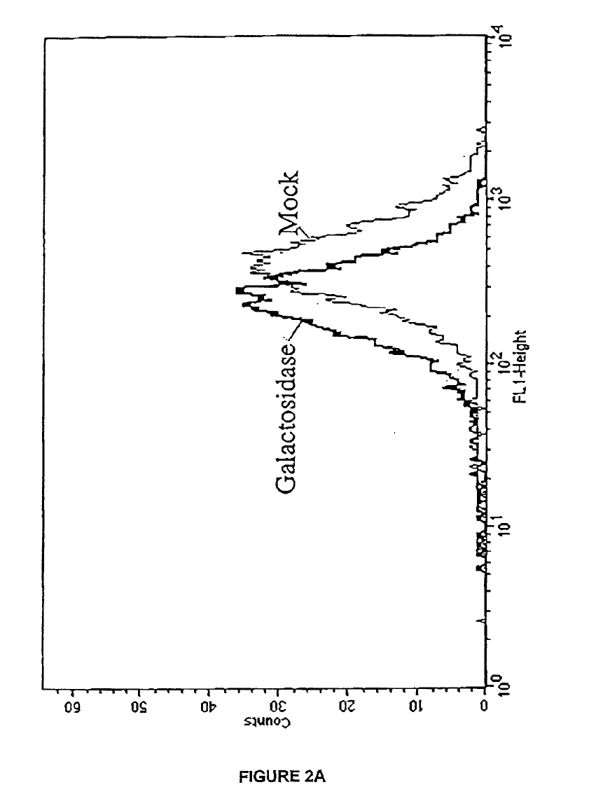

The constructs generated and used in this example are shown in FIG. 1. Standard DNA procedures were performed according to Sambrook et al. The human α-galactosidase cDNA, in the vector pAsc.8, was a generous gift from Dr. R. J. Desnick (Mount Sinai School of Medicine, New York) and was subcloned into pcDNA1(Invitrogen) to produce the plasmid pHUG (FIG. 1) as described previously (Osman 1997). Furin sequences were generated as follows; a 597 bp fragment from exon 8 of human furin (Roebrock 1986) was amplified by PCR from human genomic DNA using oligonucleotide primer pair MO123 5′TATAGCACCGAGAATGACGTG3′ (nt 2737-2757) (SEQ ID NO: 1) and MO124 5′CCAAACCCAGTCCCAAGATAA3′ (nt 3313-3333) (SEQ ID NO: 2). A second step PCR amplified an internal 260 bp fragment consisting of the furin transmembrane and cytoplasmic coding sequence including the stop codon using the primer pair GTM7 5′CATGAATTCGAGGTGGTGGCCGGCCTCA3′(nt 3001-3019 plus EcoRI site underlined) (SEQ ID NO: 3) and GTM4 5′CATCTGCAGTCAGAGGGCGCTCTGGTCTTT3′ (nt 3217-3243 Plus a Pstl site underlined and stop codon in italics) (SEQ ID NO: 4). This was subcloned by TA cloning (Holtan and Graham, 1991) into the pMosblue vector (Amersham) allowing checking of the fragment by sequencing in both directions using the ABI automated sequencer (ABI PRISM, Applied biosystems Inc. Foster city Calif.). An internal Pstl site was determined within the fragment and therefore for the purpose of further subcloning, the fragment was excised from the vector, exploiting the Xbal site of the pMosblue vector (24 nt 3′ from the GTM4 Pstl site). A second internal 211 bp fragment containing furin transmembrane and 5′ flanking region was amplified using the primer pair GTM3 (described above) and GTM8 5′CGTCTAGATCATCAGCGCAGCTGCAGGACC3′ (nt 3060-3075, Xbal site underlined and stop codon in italics) (SEQ ID NO: 5). Chimeric constructs were made as follows. The CD7 stalk sequence (147 bp) was generated by PCR from pCD7 in CDM8 (Aruffo and Seed 1987) using the primer pair GTM5 5′CATGGATCCCAAGGATGGCACAGATGC3′ (nt 895-915 plus BarnHl site underlined) (SEQ ID NO: 6) and GTM6 5′CATGAATTCTCCCTCCCGTCTCCGAC3′ (nt 985-1004 plus EcoRI site underlined) (SEQ ID NO: 7). The PCR product was digested with BamHI/EcoRI, purified and then ligated into BamHI/EcoRI-digested pHUG to produce the intermediate plasmid pHUGCD7. The plasmid pHUGCD7Fur (FIG. 1) was generated by ligating the EcoRI/Xbal-digested 260 bp furin fragment into EcoRI/Xbal-digested pHUGCD7. The plasmid pHUGTFur (FIG. 1) was produced by ligating purified BamHI/XbaI-digested 211 bp furin fragment into BamHI/Xbal-digested pHUG. The sequences of all chimeric constructs were confirmed by sequencing both strands using the ABI automated sequencer (ABI PRISM, Applied biosystems Inc. Foster city Calif.). For the purpose of producing stable expressing PIECs of HUGCD7Fur and α-galactosidase, HUGCD7Fur was subcloned, HindIlI/Xbal, into pCDNA1(Neo) and for α-galactosidase, the pAsc.8 vector containing the α-galactosidase cDNA was used.

Transfections and Immunofluorescence.

COS-7 cells were maintained in Dulbecco's Modified Eagles Medium (DMEM) (Cytosystems Pty. Ltd. Castle Hill, NSW, Australia) and were transfected using the DEAE-Dextran transfection method as described previously (Osman 1997). PIECs were maintained in Dulbecco's Modified Eagles Medium (DMEM) and were transfected using CaPO₄ transfection method as described (Wigler 1979). The transfected cells were selected by growing them in DMEM containing G418 at 2 mg/ml for 3 weeks, a number of clones were selected based on their reduction in cell surface Galα(1,3)Gal and α-galactosidase enzyme activity. P815-Py cells were maintained in DMEM and were transfected by electroporation using a Gene Pulser II apparatus (Biorad Laboratories Pty. Ltd. Regents Park, NSW, Australia). Briefly 5-20 μg DNA was added to cuvettes containing 5×10⁶ cells resuspended in phosphate-buffered sucrose (27 mM Sodium Phosphate pH 7.5, 150 mM Sucrose), samples were mixed, pulsed and added to 7.5 ml of fresh medium. 48h later transfected cells were examined for protein expression by FACS analysis on a FACSCalibur flow cytometer (Becton Dickinson Immunocytometry Systems, San Jose, Calif.). Cell surface protein expression was quantitated using Quantum™25 fluoresceinated microbeads (Flow Cytometry Standards Corp., San Juan, PR). Results are from at least five experiments.

Transfected cells were grown in 4 well plastic chamber slides and were fixed using a pH-shift/formaldehyde fixation (Pawley 1995). In some experiments, cells were treated with brefeldin A (10 μg/ml) for 10 min at 37° C. or cycloheximide (150 μg/ml) for 2h at 37° C. Fixed cells were washed with phosphate-buffered saline PBS/0.5% BSA and permeabilized with 0.1% TritonX-100 or 0.1% NP-40 in PBS. For indirect immunofluorescence the cells were exposed to primary antibodies in PBS/0.5% BSA for 30 min on ice. Cells were washed twice with PBS/0.5% BSA, incubated with secondary antibody in PBS/0.5% BSA for 30 min on ice, washed, air-dried and mounted in Prolong Antifade (Molecular Probes Inc. Eugene, Oreg.) and visualized by an Optiscan krypton/argon laser confocal microscope (Optiscan Pty. Ltd. Notting Hill, VIC, Australia). Excitation wavelengths of 488 nm and 568 nm were used for fluorescein and Texas Red-X fluorescence respectively.

α-galactosidase Assays.

Lysates from transfected cells were assayed for α-galactosidase activity using 4-methylumbelliferyl-α-D-galactopyranoside (Osman 1997) and protein was estimated using Bradford Assay. The results are from at least three experiments.

### RESULTS

### Localization of α-galactosidase and quantitation of its effects on Galα(1,3)Gal

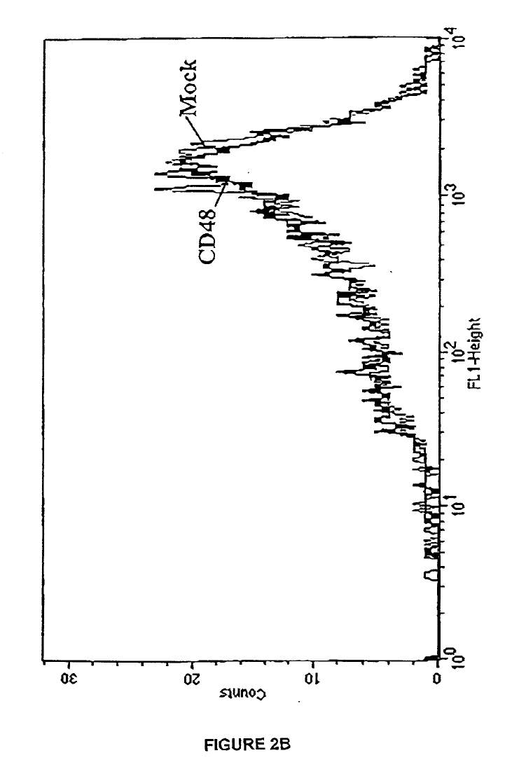

Previously, the inventors have demonstrated that overexpression of α-galactosidase in vivo in cell lines or transgenic mice decreases the level of cell surface Galα(1,3)Gal (Osman 1997). In this example, the inventors quantitated the reduction by transfecting the human α-galactosidase cDNA (pHUG) into the Galα(1,3)Gal⁺ mouse mastocytoma cell line P815py (FIG. 2A and Table I). Forty-eight hours after transfection cells were stained with FITC conjugated IB4 and the cell surface expression of Galα(1,3)Gal was quantitated by flow cytometry. Calibrated fluorescent beads were run in parallel with cell samples to provide an accurate quantitation of the level of Galα(1,3)Gal, α-galactosidase reduced the level of cell surface Gala(1,3)Gal by 31±6% (FIG. 2A and Table I) while cells transfected with a control cDNA, encoding the cell surface antigen CD48, did not significantly alter levels of Galα(1,3)Gal (FIG. 2B and Table I). These results confirm the previous studies and provide a more quantitative assessment of the ability of α-galactosidase to decrease the level of cell surface Galα(1,3)Gal.

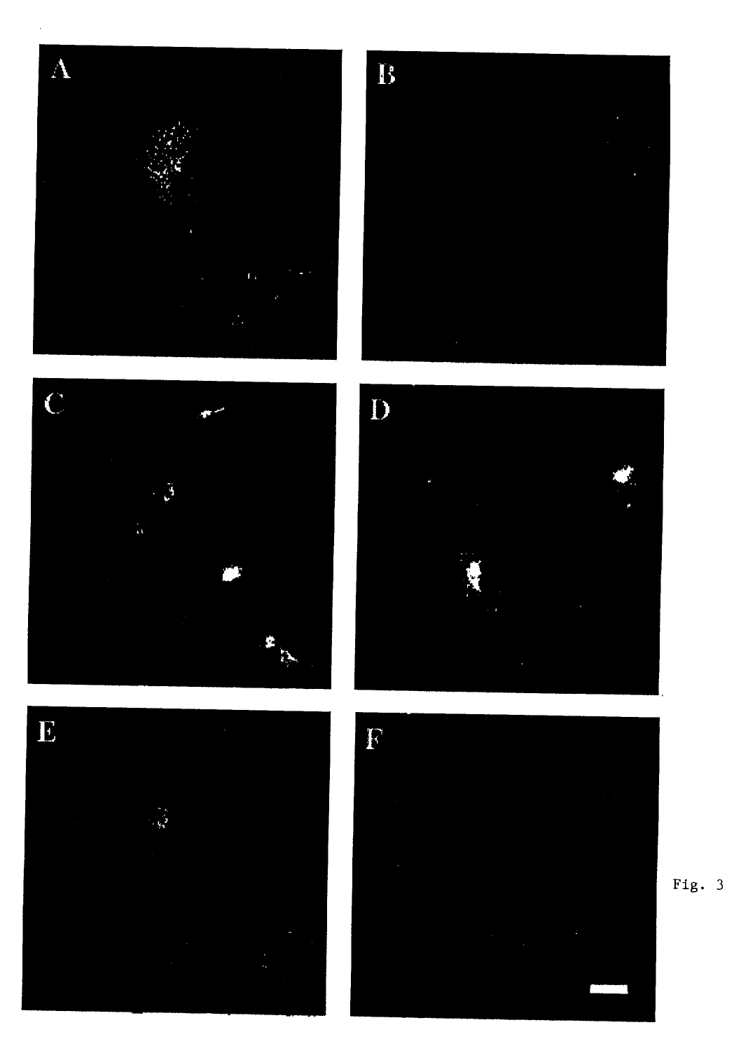

The cellular location of α-galactosidase was determined after fixation and permeabilization of transfected COS-7 cells and was visualized with anti-α-galactosidase antibody and examined by confocal microscopy. Cells expressing α-galactosidase showed predominantly punctate intracellular staining (FIG. 3A) characteristic of the lysosomal staining reported previously (loannou). Mock-transfected cells showed very weak background staining (FIG. 3B). Staining transfected cells for the TGN marker γ-adaptin, using an anti-γ-adaptin antibody (FIG. 3C) showed perinuclear foci of stain typical of Golgi staining. A small amount of coincident perinuclear staining of α-galactosidase and γ-adaptin can be seen in double-stained α-galactosidase-transfected cells (i.e. staining for α-galactosidase and γ-adaptin (FIG. 3E)). The small amount of coincident staining reflects an overlap in distributions and can be explained by two effects One, α-galactosidase is in the TGN on route to the lysosomes by the mannose-6-phosphate (M6P) pathway. Two, overexpression of α-galactosidase can quench the M6P pathway and newly synthesized α-galactosidase is retained in endosomes and the Golgi (loannou) and consequently in both cases can co-localizing with γ-adaptin. These results demonstrate that overexpression of α-galactosidase reduces the level of Galα(1,3)Gal significantly when the majority of α-galactosidase resides in lysosomes.

Targeting α-galactosidase to alternative intracellular locations.

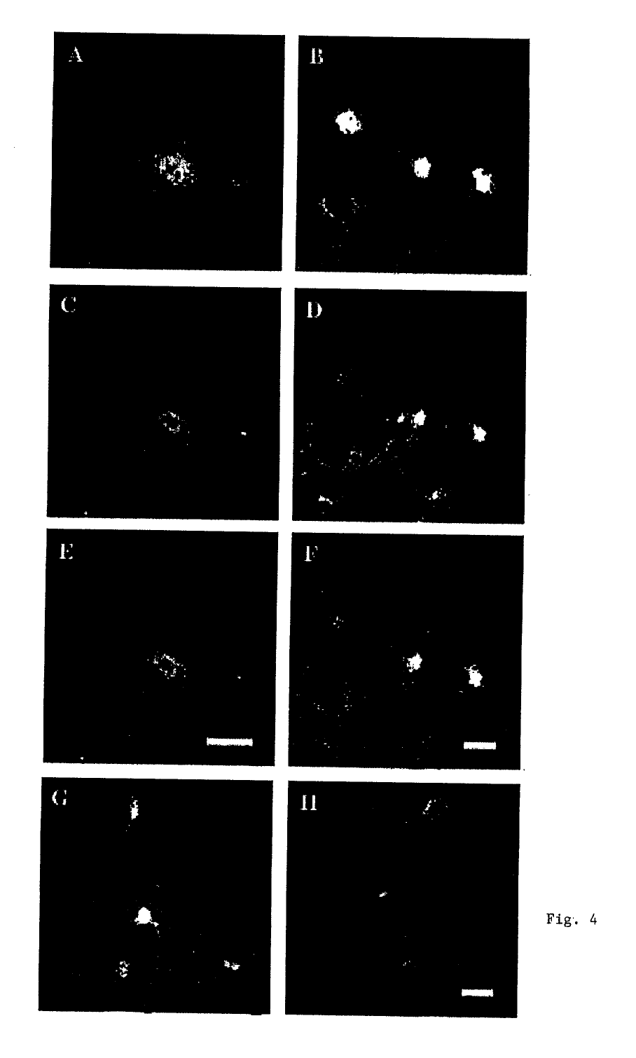

To test the hypothesis that α-galactosidase could be redirected to intracellular destinations other than the lysosomes and remain active, the inventors generated a series of chimeric Transgenes (FIG. 1). Initially, the inventors tested redirecting α-galactosidase to the Golgi complex by attaching a trans Golgi anchor based on carboxy terminal sequences of the human subtilisin protein, furin (Bosshart). Targeting signals within the furin sequence have previously been shown to localize the majority of furin to the TGN and a small proportion to the cell surface (Teuchert, and Molloy). The HUGCD7Fur chimeric construct was generated by attaching a 49 aa CD7 stalk, and the entire furin membrane spanning region and cytoplasmic domain (86 aa) to the carboxy terminal of α-galactosidase (FIG. 1). To determine the effectiveness of this chimera to redirect α-galactosidase to the TGN, the inventors performed immunofluorescent staining of COS-7 cells expressing the HUGCD7Fur transgene. Fixed, permeabilized cells were incubated with antibodies to α-galactosidase or to other Golgi markers. Immunofluorescence staining of transfected cells revealed distinct distribution patterns of α-galactosidase (FIG. 3A) and the HUGCD7Fur chimera (FIG. 4A). As noted above, α-galactosidase staining was seen as diffuse punctate staining throughout the cytoplasm (FIG. 3A) indicative of lysosomal and endosomal staining (loannou). In contrast, HUGCD7Fur staining was localized in a compact juxtanuclear region (FIG. 4A) characteristic of the Golgi apparatus. Localization to the trans Golgi was confirmed by double labeling for the TGN markers γ-adaptin (FIG. 4B) or wheat germ agglutinin (WGA)(FIG. 4E). Staining of HUGCD7Fur was coincident with staining for γ-adaptin (FIG. 4C). Similarly, coincident staining was seen with HUGCD7Fur (FIG. 4D) and WGA (FIG. 4E) with extensive overlap of the two staining patterns in the golgi region of the cell(FIG. 4F). WGA does also show significant cell surface staining not shown for HUGCD7Fur (FIG. 4), because it binds to carbohydrate epitopes not only in the Golgi but also on the cell surface

The pattern of staining the inventors observed for HUGCD7Fur was unaltered after treating the cells with cycloheximide (FIG. 4G) which blocks protein synthesis, allowing a look only at mature localized protein. This demonstrates that the observed staining pattern was not due to a transition state of the protein as it trafficks through the TGN to its destination site, internally/externally, in the cell (e.g. for instance, cell surface proteins trafficking via the Golgi) As expected γ-adaptin also retains its perinuclear staining after cycloheximide treatment (FIG. 4G). Treating the transfected cells with brefeldin A (BFA), a drug that separates the golgi and TGN, causing a redistributing of the golgi back into the ER and the TGN staying as a perinuclear foci close to the microtubule organizing center (Reaves & Banting 1992), did not alter the distribution of HUGCD7Fur, retaining its perinuclear staining pattern. This suggests that the chimera is localizing in the TGN and is similar to the published distribution for furin where it has been observed that furin can co-localize with the TGN marker, TGN 38, after the same treatment (Schafer 1995). The Texas-X-red staining of γ-adaptin can be seen to be dispersed following treatment with BFA, as described previously for this component of the AP1 adaptin complex, and is caused by a loss of recruitment of the AP1 complex to the cytosolic surface of the TGN (Molloy 1994). These results clearly demonstrate that α-galactosidase can be redirected within the cell to the Golgi apparatus, specifically the TGN, by attaching the Golgi targeting signals contained within the cytoplasmic domain of furin.

The HUGCD7Fur chimera contains a functional α-galactosidase domain.

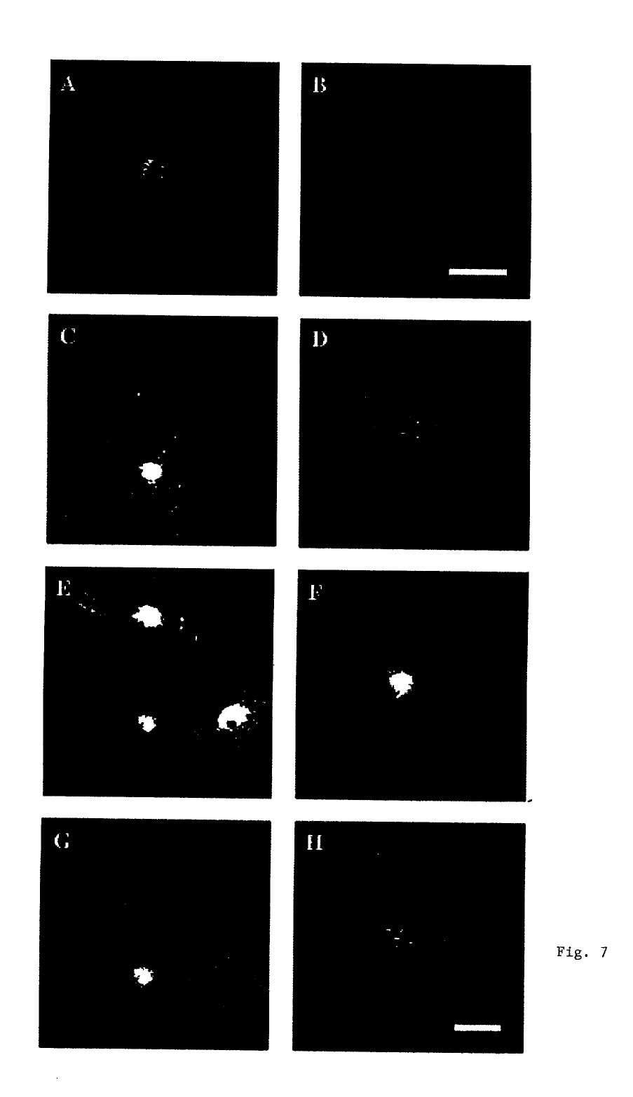

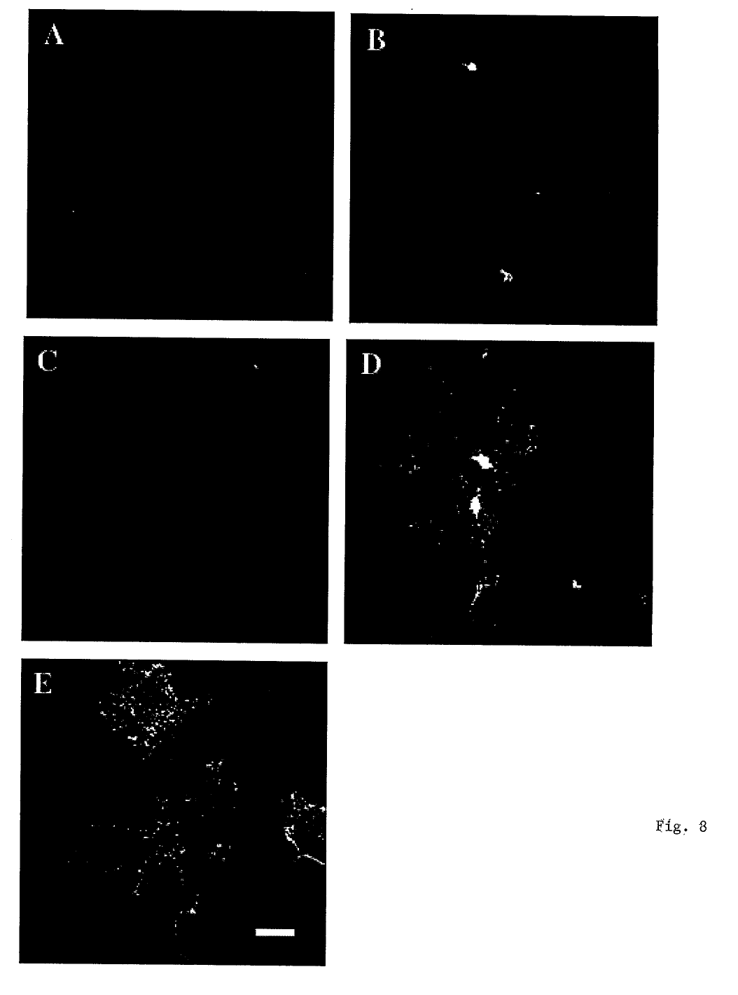

To determine whether the HUGCD7Fur chimera's α-galactosidase domain was functional the inventors looked at transfected COS-7 and stable transgene expressing, pig intestinal endothelial cell line (PIECs). For both cell lines, α-galactosidase function was assayed from cell lysates to quantitate enzyme activity. In the COS-7 cells lysates from mock transfected cells measured 395 nmoles/mg protein/h and lysates from cells expressing HUG 991 nmoles/mg protein/h, HUGCD7Fur (HCF) 1681 nmoles/mg protein/h and HUG-TFur 1317 n/moles/mg protein/h (i.e. the chimeric expressed transgene had greater activity than controls). Lysates from control transfections with CD48 had 59±0.1 nmoles/mg protein/h α-galactosidase activity confirming that activity above mock activity was restricted to the chimeric transgene. The PIECs were transfected with α-galactosidase and HUGCD7Fur selected for stable expression, a number of clones for each transgene were characterized One clone representing each transgene is shown in this analysis. Parental PIECs give a background enzyme activity of 62±3.0 nmoles/ug protein/hr, both HUGCD7Fur and α-galactosidase give comparable increases in activity of 136±6.9 149±7.8 nmoles/ug protein/hr respectively (FIG. 7). Together these results clearly demonstrate that the chimeric construct HUGCD7Fur has functional α-galactosidase domain despite the addition of a transmembrane region and cytoplasmic tail, containing TGN localization signals, to the carboxy terminus.

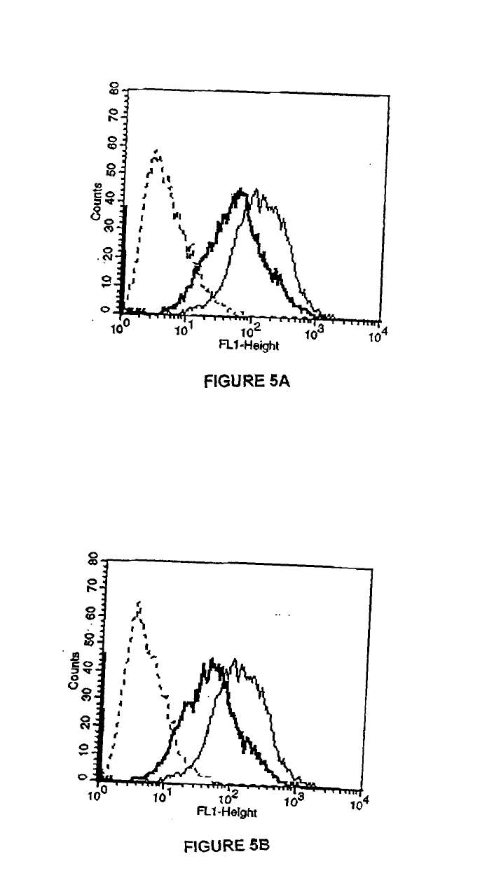

A comparison was also made of the efficiency of removing cell surface Galα(1,3)Gal in two cell lines expressing endogenous Galα(1,3)Gal, PIECs and P815-py. The degree of Galα(1,3)Gal expression was determined by labeling with IB4 conjugated to FITC and quantitating surface fluorescence by flow cytometry. In the case of the P815-py transfectants, the MESF was determined using calibrated fluorescent beads (Table 1). For the PIECs, HUGCD7Fur and α-galactosidase reduced the level of Galα(1,3)Gal to similar degrees, approximately 50% shift down in fluorescence when compared to parental PIECs (FIGS. 5A&B). The P815-py cell results confirmed what was observed for the PIECs with HUGCD7FUR reducing the level of Galα(1,3)Gal by 25±8% (Table 1) i.e. a reduction in Galα(1,3)Gal approximately the same as we observed by the expression of α-galactosidase.

Redirection of α-galactosidase to the cell surface.

In addition to redirecting α-galactosidase to the TGN, the inventors generated a second chimeric construct HUGTFur (FIG. 1) to redirect α-galactosidase activity to the cell surface. The strategy was based on previously published reports of retaining furin at the cell surface by removal of its retention and retrieval signals contained within the furin carboxy terminal sequence (Bosshart 1994, Schafer 1995). COS-7 cells were transfected with HUGTFur and the cellular distribution of the protein was examined by confocal microscopy. Internal staining of permeablised HUGTFUR expressing cells (FIG. 7C), using an anti-α-galactosidase antibody, showed a perinuclear staining pattern of the Golgi Apparatus similar to that shown for HUGCD7FUR (FIG. 4A). The TGN marker γ-adaptin (FIG. 7D) was used to confirm that the distribution was to the TGN and its staining pattern was coincident with HUGTFur (FIG. 7E). When the transfected cells were treated with cycloheximide, HUGTFur's perinuclear staining was lost (FIG. 7F) while the γ-adaptin staining was retained (FIG. 7G). No coincident staining of γ-adaptin and HUGTFur was present (FIG. 7H) demonstrating that localization in the TGN of HUGTFur is a transition state. When the surface of these cells is stained it was found that before and after cycloheximide treatment, the HUGTFUR was localized to the cell surface (FIGS. 7A&B). These results, internal and cell surface staining, indicates that HUGTFur is trafficked through the TGN and localizes on the cell surface, like most cell surface expressed proteins. This is in agreement with previous reports of similar furin constructs in which the entire cytoplasmic domain has been deleted (the retrieval and retention signals) (Schafer 1995). The efficiency of this chimera, HUGTFur, to decrease the level of Galα(1,3)Gal on the cell surface of transfected p815-py cells was 5-10% less effective than HUGCD7Fur (Table 1) i.e. HUGTFur decreased Galα(1,3)Gal 14±4%. However, despite the limitations of using this construct to decrease the level of Galα(1,3)Gal, it is clear that the strategy employed to redirect α-galactosidase was effective.

HCF is able to recycle from the cell surface to the TGN.

Furin can cycle in a rapid TGN→Cell surface→TGN manner. To confirm whether the furin retention and retrieval signals contained within our HUGCD7Fur cytoplasmic tail allowed a similar cycling pattern to occur, COS-7 cells expressing HUGCD7Fur were exposed to the anti-α-galactosidase antibody in culture for 2 hrs. The cells where fixed, permeablised and stained with a secondary antibody. For Mock transfected cells, secondary antibody alone (FIG. 7C), irrelevant primary antibody (FIG. 7D) and α-galactosidase transfected cells (FIG. 7E) only non-specific stain pattern were present. In the case of HUGCD7Fur (FIG. 7F) a specific staining pattern was observed with vesicular staining within the cytoplasm and the characteristic perinuclear staining pattern of the TGN. HUGTFur demonstrated only cell surface staining as would be expected considering this transgene lacks the localization signals necessary for TGN retrieval from the cell surface and TGN retention. These results show that the furin cytoplasmic tail signals in HUGCD7Fur are sufficient to transport and retrieve HUGCD7Fur to and from the cell surface.

HUGCD7FUR is effective as a combined approach with the fucosyl transferase (HT) in removing Galα(1,3)Gal.

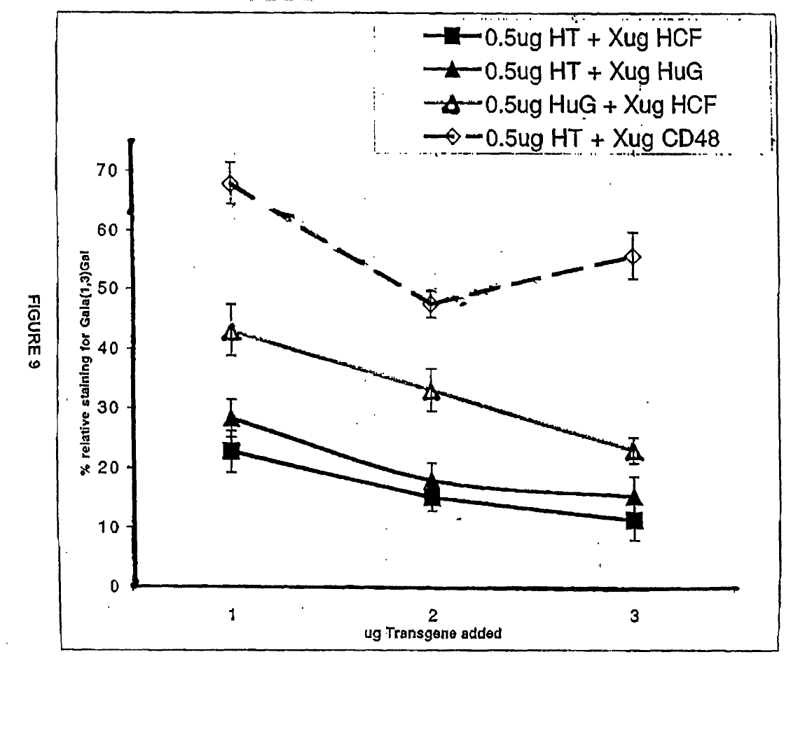

It has been previously reported that α-galactosidase can reduce Galα(1,3)Gal in an additive manner when co-expressed in COS-7 cells with the α(1,2) fucosyl transferase (HT) and that this combination was more effective than the expression of each transgene alone (Osman). It was therefore postulated that a synergistic effect could occur also with the co-expression of HUGCD7Fur with HT and allow a greater reduction in Galα(1,3)Gal. To answer this, the inventors co-transfected the transgenes into COS-7, using a constant concentration of α(1, 3) galactosyl tranferase (GT) and HT with increasing concentrations of HUGCD7Fur, α-galactosidase, control plasmid CD48 (FIG. 9). The inventors also wanted to determine whether an additive effect could be seen for the co-expression of α-galactosidase and HUGCD7Fur and so transfected constant amounts of GT and α-galactosidase with increasing concentrations of HUGCD7FUR (FIG. 9). 48 hrs later the transfected cells were surface stained with IB4 conjugated to FITC, fixed and the number of positive verses negative cells counted by fluorescence microscopy, to determine the percentage of positive staining cells. The percentage was related back to GT transfected cells alone (transfection efficiency) and this taken as being 100 percent positive HUGCD7FUR or α-galactosidase when co-expressed only with GT reduce Galα(1,3)Gal in a linear, concentration dependent manner (FIG. 9). When they are expressed with HT this reduction is greater and is the combined additive effects of both transgenes (FIG. 9). When α-galactosidase is expressed with HUGCD7Fur an additive effect also occurs although not to the extent of when HT is expressed. HT is a much more effective transgene in reducing cell surface Galα(1,3)Gal. CD48 (FIG. 9), as expected, does not reduce the expression of Galα(1,3)Gal demonstrating that the observed reductions in Galα(1,3)Gal is not due to overexpresion of the transgenes causing a reduction in GT expression and therefore a reduction in synthesized Galα(1,3)Gal. This confirms that HUGCD7Fur can be an effective strategy in reducing cell surface expression of Galα(1,3)Gal when co-expressed with HT.

TABLE 1
Quantitation of Gal α(1,3) Gal

| | mean equivalent soluble | % reduction in |
|---|---|---|
| Construct transfected | Fluorochrome (MESF) | Gal α(1,3) Gal |
| Mock (pCDNA) | 402794 | 0 |
| α-galactosidase | 294132 | 31 ± 6 |
| HUGCD7Fur | 302095 | 25 ± 8 |
| HUGTFur | 346400 | 14 ± 4 |
| CD48 | 371552 | 8 ± 5 |

Cell surface Galα(1,3)Gal expression of Galα(1,3)Gal⁺ mouse mastocytoma cell line P815py transfected with each construct as indicated was quantitated using fluorosceinated microbeads. Results are expressed as mean±SEM from at least five experiments.

### DISCUSSION

The central aim of this example was to establish whether a lysosomal glycosidase could be redirected within a cell whilst maintaining functional activity. α-galactosidase exists, in its usual state, as a soluble homodimer in the lysosome (loannou). From the inventors' results, it is clear that attaching a membrane anchoring region to the carboxy end of α-galactosidase has not affected its glycosidase activity nor presumably has it been detrimental to its active tertiary conformation. Enzyme activity was maintained irrespective of redirection of the enzyme to either the TGN or to the plasma membrane.

Using α-galactosidase antibody as a specific probe enabled the steady-state distribution of the chimeric α-galactosidase enzymes HUGCD7Fur and HUGTFur to be established. HUGCD7Fur was predominantly localized to the TGN as demonstrated by the co-localization studies with TGN markers γ-adaptin and WGA (FIG. 4). This location indicates that the signals contained within the furin transmembrane and cytoplasmic tail sequences are the dominant targeting signals in these chimeras while the lysosomal trafficking signals contained within the α-galactosidase sequence have been overidden. The cytoplasmic tail of furin has previously been well documented to contain sequences to target the majority of furin to the TGN (Molloy 1994, Bosshart 1994, Schafer 1995, Jones 1995, Shapiro 1997) and a small amount to the plasma membrane (Bosshart 1994, Schafer 1995). Furin sequence between Pro₇₇₂ and Arg₇₈₄ contains an acidic cluster and a casein kinase II consensus sequence that together are used to localize furin to the TGN (Schafer 1995, Jones 1995), TGN retention signal, and a tyrosine based signal Y₇₆₂KGL₇₆₅ (SEQ ID NO: 8) that is believed to provide endosomal localization (Bosshart 1994), the TGN retrieval signal. Furthermore, co-localization studies with Golgi markers γ-adaptin (Molloy 1994) and TGN 38 (Bosshart 1994, Schafer 1995, Shapiro 1997) and immunoelectron microscopic studies (Molloy 1994, Bosshart 1994, Schafer 1995) confirmed that furin is localized to the Golgi and, more specifically, TGN tubules and clathrin coated buds and vesicles. Confocal microscopy results for HUGCD7Fur are in agreement with all of the published data that indicates an intact furin cytoplasmic tail is a requirement and sufficient for TGN localization.

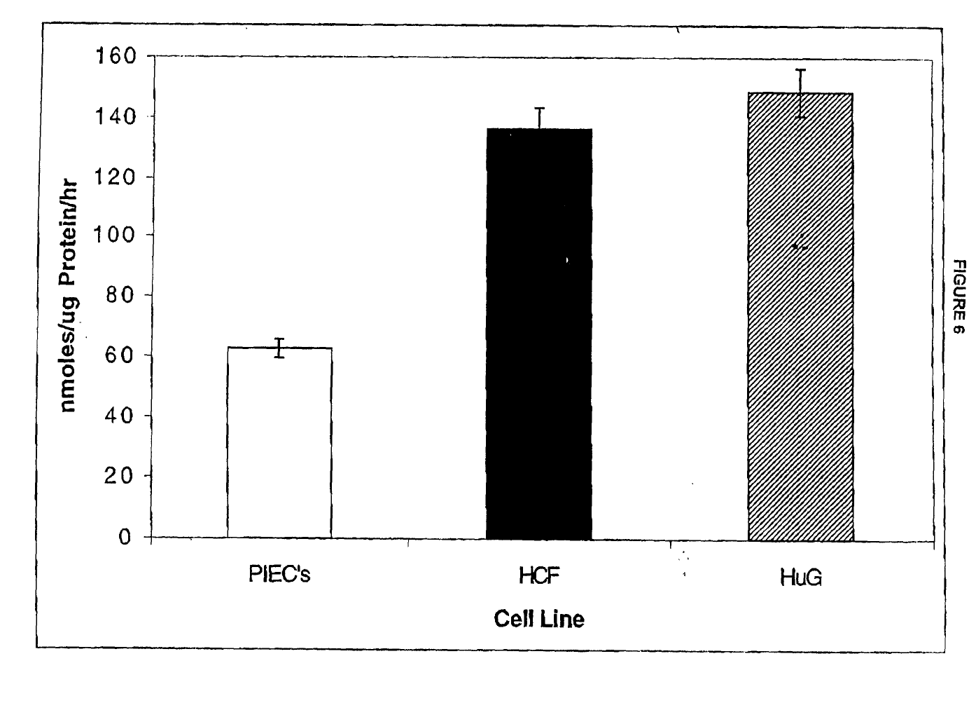

Deletion of the furin cytoplasmic tail is known to cause a dramatic change in the steady-state distribution of furin with cytoplasmic tail deletion constructs being delivered to the plasma membrane and primary vesicles of the endosomal/lysosomal system (Bosshart 1994, Schafer 1995). Cytoplasmic tail deletion mutants are no longer retrieved from the cell surface as shown by an absence of antibody internalization by such transgenes (Molloy 1994,Jones 1995) and is a result of the removal of the retrieval signal normally contained in the furin cytoplasmic tail. The HUGTFur chimera, lacking the entire furin cytoplasmic domain, showed characteristics typical of cytoplasmic tail deletion constructs (i.e. HUGTFur was localized at the plasma membrane (FIG. 6) and a lack of antibody internalization (FIG. 7G)). Again, the remaining signals of the furin transmembrane domain apparently dominated the targeting of this construct with the α-galactosidase signals playing a secondary or insignificant role. α-galactosidase, as a lysosomal hydrolase, is targeted to the lysosomes via recognition of a mannose-6-phosphate (M6P) moiety on high mannose oligosaccharides on the hydrolase (Kornfeld, 1986). The M6P signal is attached to the high mannose oligosaccharide on Asn₂₁₅ in α-galactosidase as has been determined by the deletion of this site (loannou 1992). From the inventors' results it is clear that attaching sequences to the carboxy terminus of α-galactosidase was sufficient to disrupt or override the normal lysosomal targeting signals: neither HUGCD7Fur or HUGTFur localized to the lysosomes in contrast to wild-type α-galactosidase. Despite the domination of the furin targeting signals, the activity of the chimeric transgenes was not affected with both transgenes having measurable enzyme activity and with a decrease in Galα(1,3)Gal resulting from expression of either transgene. While not wishing to be bound by theory, it is unlikely that the CD7 stalk region included in the HUGCD7Fur chimera contributed to the targeting of this transgene in view of the “furin-like” localization behavior of the construct and the extended structure of the CD7 stem which contains four repeats with a high proportion of Pro, Ser and Thr residues. This type of extended structure is thought to provide a spacer between the transmembrane and lγ-like domains of CD7 (Aruffo and Seed 1987) and localization signals have not been found in this region. The CD7 stalk region was included in the HUGCD7Fur transgene essentially as a spacer to distance the α-galactosidase portion of the transgene away from the furin transmembrane domain. HUGCD7Fur was the inventors' first chimera and it was not clear whether adding carboxy terminal sequences to α-galactosidase would impose potential folding constraints such that enzyme activity would be compromised. It is apparent from the HUGTFur construct, which does not contain a spacer region, that the addition of this sequence was not essential to the function of our chimeric enzymes.

Using two α-galactosidase chimeras, the inventors have been able to redirect α-galactosidase activity to two intracellular sites that would not normally be expected to sustain α-galactosidase activity, namely the TGN and the plasma membrane. That these sites are foreign to the enzyme is reflected in the observation that the ability of these chimeras to reduce Galα(1,3)Gal was not significantly different to the wild-type α-galactosidase (Table I). This suggests that despite having viable enzyme in chosen locations, that it may be important to select an enzyme with the appropriate biochemical characteristics (e.g. reduced pH sensitivity) of the intended microenvironment.

Targeting α-galactosidase would also be useful in enzyme replacement therapy for patients with Fabry disease, an X-linked inherited disorder of glycosphingolipid metabolism that leads to the accumulation of ceramide trihexoside and other glycolipids that deposit in tissues and can eventually lead to organ failure. Enzyme replacement therapy has been tried for this disease (Medin), however the enzyme has a short half-life and is rapidly cleared from the circulation. Gene replacement therapy has also been undertaken in vitro using retroviral vectors with some success (Medin 1996). Extrapolating the results from the present example, it would be useful to have a gene replacement therapy based on a chimeric α-galactosidase containing endosomal targeting signals and retrieval signals of furin or other targeting signals attached to a pH modified α-galactosidase. This would allow the degalactosylation of accumulated glycolipids by an active chimeric α-galactosidase such that the degradation of these glycolipids can resume and the amount of intracellular deposits reduced.

### Example 2

a) Generation of the HCF transgene and transgenic line

HCF is a chimeric transgene designed with the cDNA encoding the catalytic domain of HUG attached to the cDNA encoding for the transmembrane region and cytoplasmic domain of furin, a CD7 stalk cDNA region was placed as a spacer between the cDNA of HUG and the cDNA Furin. The furin cytoplasmic tail was chosen to redirect α-galactosidase because it is a compatible type-1 protein and contains well characterised TGN localization signals. It is these signals which have been exploited in HCF, with the rational that it will be possible to redirect α-galactosidase which normally is localized in the lysosomes of cells, to reside in the TGN, where it is in closer proximity to Galα(1,3)Gal epitopes before they reach the cell surface.

HCF was placed under the H2Kb promoter. This was achieved by creating terminal Notl/Nrul restriction enzyme site on the coding region of the HCF transgene and ligating it into the H2Kb cassette. The H2Kb cassette consists of the H2Kb promoter and the first 11 exons of the H2Kb coding region. This procedure allows the replacement of exon 1 of the H2Kb gene for that of HCF and therefore the transgene will be expressed instead of the H2Kb coding region (FIG. 1).

The H2Kb HCF transgene was injected into the pronucleus of mouse embryos and the embryos implanted into pseudo pregnant mice. The resultant progeny were screened for the integration of the transgene. The screening involved southern blots of the genomic DNA from these transgenic mice probing with CD7-Fur portion of the transgene to determine integration into the mouse genome and functionally by MUG assay on spleen lysates.

b) Generation of transgenic mice

A PCR fragment of the entire HCF coding region was generated with Nrul/Notl restriction enzyme site ends. This PCR product was digested with Nrul & Notl, purified and directionally ligated into the Nrul/Not l site in exon 1 of the murine H2Kb gene replacing the start codon of the H2Kb gene with that of the transgene. The generated H2Kb HCF transgene was microinjected into the pronuclear membrane of SJLxBI/6 zygotes at a concentration of 2-5 ng/ul. The zygotes were implanted into pseudo pregnant mice and the resultant progeny screened for the transgenes either by Southern blot, Southern, Immunoflourescence of cell surface carbohydrates, or by enzyme activity of α-galactosidase.

To produce a homozygous line of the HCF, homozygous transgenic mice were brother/sister mated for three generations, screening each generation by Southern to select homozygous mice for the next generation.

c) Expression of Galα(1,3)Gal

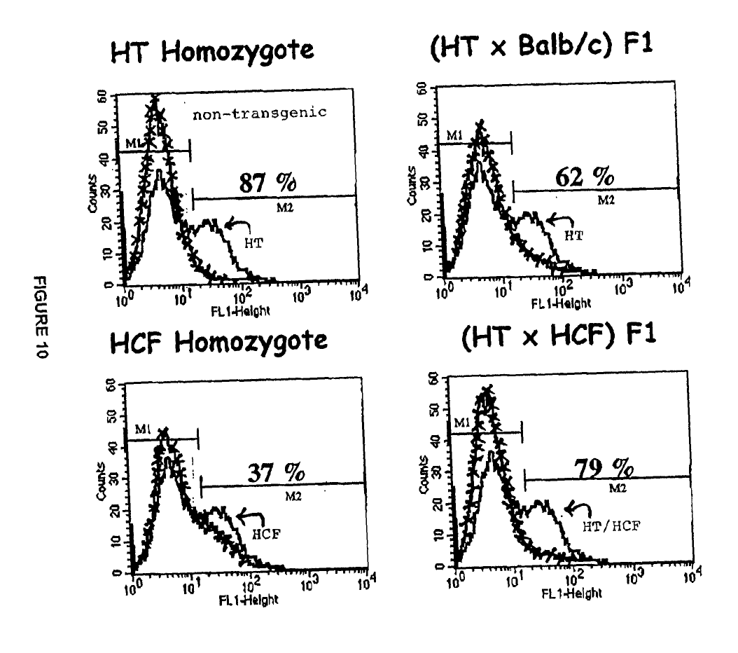

Epitope expression in splenocytes was determined by IB4 labelling with IB4 conjugated to FITC as described above. FIG. 10 shows that in animals homozygous for HCF, there was a 37% reduction in fluorescence compared with non-transgenic mice. HT homozygote mice gave an 87% reduction and HT hemizygote gave a 62% reduction. In F1 hybrid mice with HT (Cohney 1997), there appeared to be a synergistic effect as a 79% decrease in fluorescence was observed. These results show that transgenic mice expressing HuGCD7Fur or HCF had greater α-galactosidase activity than non-transgenic mice and a reduction in Galα(1,3)Gal, while hybrid mice with HT showed even greater reduction.

1. A nucleic acid construct encoding a peptide with hydrolase activity and a heterologous localization signal which locates the peptide, upon expression of the construct in a cell, to a cellular location proximal to the cellular site of a glycosylated agent or a glycosylating agent.

2. The construct of claim 1, wherein said localization signal locates the expressed peptide to a cellular location proximal to the cellular site of a newly synthesized molecule comprising at least one sugar moiety.

3. The construct of claim 1, wherein said localization signal locates the expressed peptide to a cellular location proximal to the cellular site of a newly synthesized molecule comprising terminal galactose residues.

4. The construct of claim 3, wherein said terminal galactose residues are α-linked gal.

5. The construct of claim 1, wherein said localization signal locates the expressed peptide to a cellular location wherein said peptide inhibits production of a glycosylated agent.

6. The construct of claim 1, wherein said localization signal locates the expressed peptide to a cellular location proximal to the cellular site of α1,3 galactosyl transferase.

7. The construct of claim 1, wherein said localization signal locates the expressed peptide to the cell surface, an endosome, a lysosome, the Golgi or the transgolgi network.

8. The construct of claim 1, wherein said localization signal is derived from a Type I Golgi protein.

9. The construct of claim 1, wherein said localization signal is derived from furin.

10. The construct according to claim 1, wherein said peptide is an α-galactosidase or a functional portion thereof.

11. The construct according to claim 10, wherein said galactosidase or functional portion thereof removes the terminal, α-linked galactose from Galα(1,3)Gal.

12. The construct of claim 11, wherein said localization signal is derived from a Type I Golgi protein.

13. The construct of claim 11, wherein said localization signal is derived from furin.

14. The construct of claim 10, wherein said localization signal locates the expressed galactosidase or functional portion thereof to the cell surface, an endosome, a lysosome, the Golgi or the transgolgi network.

15. A method of locating a peptide with hydrolase activity to a cellular location proximal to the cellular site of a glycosylated agent or a glycosylating agent within a cell, said method comprising expressing in the cell a nucleic acid construct according to any one of claims 1 to 14.

16. A non-human transgenic animal, wherein cells of said animal include and express the construct according to any one of claims 1 to 14.

17. The animal of claim 11, wherein said animal is a pig.

18. An organ isolated from the animal of claim 16, said organ being suitable for xenotransplantation.

19. An organ isolated from the animal of claim 18, said organ being suitable for xenotransplantation

20. A cell including a construct according to any one of claims 1 to 14.

21. A method of modulating hydrolysis of a glycosylated product expressed in a cell, said method comprising introducing into said cell the construct of any one of claims 1 to 14.

22. A method of alleviating a disease or condition in a patient caused by the presence of undesirable glycosylated products, said method comprising introducing into cells of the patient the nucleic acid construct of any of claims 1 to 14 such that the construct is expressed and said localization signal locates the expressed peptide to a cellular location proximal to the cellular site of said glycosylated products or a glycosylating agent producing said glycosylated products such that said hydrolase activity of the expressed peptide reduces the glycosylation on the glycosylated products.

23. The method of claim 22, wherein said disease is Fabry disease.


SEQUENCE LISTING (8 sequences)

SEQ ID NO: 1 - DNA, 21 bp, Artificial Sequence
  PCR primer
tatagcaccgagaatgacgtg

SEQ ID NO: 2 - DNA, 21 bp, Artificial Sequence
  PCR primer
ccaaacccagtcccaagataa

SEQ ID NO: 3 - DNA, 27 bp, Artificial Sequence
  PCR primer
catgaatccgaggtggtggccggcctc

SEQ ID NO: 4 - DNA, 30 bp, Artificial Sequence
  PCR primer
catctgcagtcagagggcgctctggtcttt

SEQ ID NO: 5 - DNA, 30 bp, Artificial Sequence
  PCR primer
cgtctagatcatcagcgcagctgcaggacc

SEQ ID NO: 6 - DNA, 27 bp, Artificial Sequence
  PCR primer
catggatcccaaggatggcacagatgc

SEQ ID NO: 7 - DNA, 26 bp, Artificial Sequence
  PCR primer
catgaattctccctcccgtctccgac

SEQ ID NO: 8 - protein, 4 aa, Artificial Sequence
  Putative endosomal localisation signal of furin
YKGL